(19) United States
(12) Patent Application Publication
(10) Pub. No.: US 2019/0175785 A1
(43) Pub. Date: Jun. 13, 2019

(54) SILK FIBROIN SYSTEMS FOR ANTIBIOTIC DELIVERY

(71) Applicant: Trustees of Tufts College, Medford, MA (US)

(72) Inventors: David L. Kaplan, Concord, MA (US); Bruce Panilaitis, Tewksbury, MA (US); Eleanor M. Pritchard, Somerville, MA (US); Fiorenzo G. Omenetto, Lexington, MA (US)

(21) Appl. No.: 16/033,650

(22) Filed: Jul. 12, 2018

Related U.S. Application Data
(63) Continuation of application No. 14/050,624, filed on Oct. 10, 2013, now abandoned.
Application No. 14/050,624 is a continuation of application No. 13/254,629, filed on Nov. 8, 2011, now abandoned.
(60) Provisional application No. 61/157,366, filed on Mar. 4, 2009.

(51) Int. Cl. A61L 27/22; A61K 47/42; A61K 9/00; C07K 14/435; A61K 9/16; A61K 9/70; A61L 27/54; A61L 27/36; A61L 15/44; A61L 15/40; A61K 45/06; A61K 31/7036; A61K 31/546; A61K 31/43

(52) U.S. Cl. CPC A61L 27/227; A61K 47/46; A61K 9/0019; C07K 14/43586; A61K 9/1617; A61K 9/1664; A61K 9/7007; A61L 27/54; A61L 27/3604; A61L 15/44; A61L 15/40; A61K 45/06; A61K 31/7036; A61K 31/546; A61K 31/43; A61K 47/42

(57) ABSTRACT

The present invention provides for silk fibroin-based compositions comprising one or more antibiotic agents for prevention or treatment of microbial contamination, methods of making antibiotic-containing silk scaffold, methods of stabilizing antibiotics in silk scaffolds, and methods for preventing or treating microbial contamination using the antibiotic-containing compositions. Various methods may be used to embed the antibiotic(s) into the silk fibroin-based compositions. The antibiotic-containing compositions of the invention are particular useful for stabilizing antibiotics, preventing bacterial infections, and for medical implants, tissue engineering, drug delivery systems, or other pharmaceutical or medical applications.

### CROSS REFERENCE TO RELATED APPLICATIONS

This application is a continuation application of U.S. patent application Ser. No. 14/050,624 filed Oct. 10, 2013, which is a continuation application of U.S. patent application Ser. No. 13/254,629 filed Nov. 8, 2011, now abandoned, which is a 35 U.S.C. 371 National Phase Entry application of International Application PCT/US2010/026190 filed Mar. 4, 2010, which designates the United States, and which claims benefit under 35 U.S.C. § 119(e) of U.S. Provisional Application 61/157,366 filed Mar. 4, 2009, the contents of each of which are incorporated herein by reference in their entireties.

### GOVERNMENT SUPPORT

This invention was made with government support under EB002520 awarded by the National Institutes of Health, and W911NF-07-1-0618 awarded by the United States Army. The government has certain rights in the invention.

### FIELD OF THE INVENTION

This invention relates to compositions for preventing or treating microbial contamination, and methods of preventing or treating microbial contamination using such compositions. The compositions of the invention exhibit superior stability, and may be used in medical implants, tissue engineering, drug delivery systems, or other pharmaceutical or medical applications.

### BACKGROUND OF THE INVENTION

Biomaterials have been developed for a variety of applications including cardiovascular and musculoskeletal implants, as substrates for tissue engineered cartilage, bone and ligaments, as viable media for cellular proliferation and drug delivery, and for directing the appropriate differentiation of human mesenchymal stem cells into specific tissues.

In evaluating the efficacy of biomaterial compositions for use in implants, tissue engineering, drug delivery or regenerative medicine, a major limitation concerns the susceptibility of these applications to microbial contamination, particularly caused by surgical site infection. Surgical site infections are the second-most common cause of nosocomial infections. Patients that develop surgical site infections are more likely to be admitted to an intensive care unit or be readmitted to the hospital, and more likely to die, than patients avoiding surgical site infection.

Antimicrobial prophylaxis provides a common and effective method of preventing microbial contamination following surgery. Current antimicrobial options have significant systemic side effects and limitations, however. For example, there are numerous inconsistencies regarding the appropriate selection, timing, and duration of administration of prophylactic antimicrobials. Additionally, the antimicrobial should be administered as near to the incision or implant area as possible to achieve the lowest surgical site infection rates. Moreover, a systemic antimicrobial approach to infection prevention often results in insufficient local concentrations of antibiotic and significantly increases the risk for surgical site infection.

For example, typical treatment of an infected abscess is draining of the wound, packing with gauze, and systemic administration of antibiotics. Abscesses formed in the presence of Staphylococcus aureus infections, however, typically develop an epithelial barrier or shell through which antibiotics fail to penetrate. Current non-degradable surgical packing materials require surgical retrieval and have no inherent antibiotic activity. Moreover, in these patients heavy systemic doses of antibiotics may damage the liver, and waste large amounts of drug. A simple system for antibiotic delivery is needed that also stabilizes the incorporated drug, restricts delivery to a specific target site to minimize cost and side-effects, while maximizing efficacy and biodegrades to avoid surgical retrieval.

Hence, there remains a need for compositions comprising a natural polymeric medium that not only offers a medically-relevant, biocompatible, and mechanically viable structure for implants, tissue repair or drug delivery systems, but also locally directs the administration of antimicrobial to the incision, implant, or the target delivery area to effectively prevent or treat an infection.

### SUMMARY OF THE INVENTION

The present invention provides for silk fibroin-based compositions for medical implants, tissue engineering, or drug delivery systems to prevent and/or treat microbial contamination. The invention further provides methods for preventing and/or treating microbial contamination by using the compositions of the invention. More specifically, the antibiotic-loaded silk fibroin systems of the present invention are biocompatible, safe, FDA-approved and degrade in vivo to nontoxic products. Antibiotic-loaded silk biomaterials can be applied to or injected into target sites, delivering antibiotics locally or regionally and avoiding systemic side-effects from large doses of antibiotics. Unlike some current surgical packing materials (e.g., gauze), silk degrades naturally over time, so surgical removal is unnecessary. Moreover, the present invention provides for remarkable antibiotic stability at a wide range of relevant temperatures. For example, antibiotic-loaded silk compositions of the present invention can be stored at room temperature, then applied or injected for sustained release at the site of infection where they subsequently biodegrade. The compositions of the present invention may be designed to deliver a large preliminary “burst” dose of antibiotics, followed by a slow, sustained release of a lower maintenance dose.

One embodiment of the invention relates to a composition comprising a silk fibroin scaffold and at least one antibiotic agent. In particular embodiments, the silk fibroin-based scaffold comprises antibiotic-loaded microspheres embedded in a porous silk fibroin matrix or gel. In some embodiments, the silk fibroin scaffold may comprise a film, slab, or comprise a three-dimensional structure such as a matrix or gel. In particular embodiments, the silk scaffold is a coating on a substrate suitable for use, for example, as a bandage or an implant. Example antibiotics in particular embodiments include cefazolin, gentamicin, penicillin, and ampicillin. In some embodiments, the antibiotic-loaded silk fibroin composition may include at least one additional agent, such as a biologic or drug. The compositions of the invention may be used for medical implants, tissue engineering, regenerative medicine, or drug delivery systems to prevent and/or treat microbial contamination. The compositions can be formulated to deliver the at least one antibiotic agent at levels exceeding the minimum inhibitory concentration (MIC) for organisms commonly found to be the cause of such microbial contamination.

Various methods may be employed to embed at least one antibiotic agent into the silk structure. For example, the antibiotic may be added to a silk fibroin solution before forming a silk scaffold (i.e., antibiotic is incorporated directly into a silk film, gel, or porous matrix); silk fibroin microspheres comprising antibiotic may be prepared, then these antibiotic-loaded microspheres may be mixed with silk solution to form silk scaffolds (such as a gel or porous matrix) into which the microspheres are embedded; or one or more antibiotic-loaded layers can be coated on silk scaffolds. The methods of the present invention may also include the step of adding an additional agent to the antibiotic-containing scaffold.

The present invention also provides for the long term storage of antibiotics and/or other agents in a silk-fibroin composition. For example, a method for preparing a long-term antibiotic storage composition comprises selecting an antibiotic, incorporating the antibiotic into a silk-fibroin solution, and forming a scaffold from the solution. The solution may be an aqueous solution or a hydrated lipid solution. An additional agent, such as a drug or biologic, may be added to the solution. The scaffold may be formed by pouring the solution onto a surface to yield a film or slab. Alternatively, the scaffold may be formed by pouring the solution into a mold or container and then dried to form a three-dimensional porous matrix. A solution may be treated to create antibiotic-containing nanoparticles or microspheres. The solution may be sonicated to form a gel. Additionally, the antibiotic-loaded microspheres may be added to another silk fibroin solution and then formed into gel or matrix. Antibiotic prepared by these methods maintain at least 75% residual activity for at least 60 days when stored at 4° C., 25° C., or 37° C.

Another embodiment of this invention relates to a method of preventing and/or treating microbial contamination at a region of an object or subject for medical implants, tissue engineering, regenerative medicine, or drug delivery systems. The method comprises contacting the region of the object or subject with a composition comprising a three-dimensional silk fibroin-based silk scaffold and at least one antibiotic agent. The contacting may be achieved by a bandage, sponge, or surgical packing material. The composition may be formulated to deliver the at least one antibiotic agent at levels exceeding the MIC for organisms such as those commonly found to be the cause of microbial contamination. For example, possible microbial contamination may be associated with a surgical site infection. In one embodiment, the surgical prophylactics such as cefazolin, gentamicin, penicillin, ampicillin, or a combination thereof, may be incorporated into the silk fibroin to prevent or treat surgical site infections.

### DESCRIPTION OF THE DRAWINGS

FIG. 1 depicts cumulative in vitro release of gentamicin in 100 μl of water from silk fibroin scaffolds embedded with antibiotic-loaded microspheres, layered with antibiotic, embedded with antibiotic directly in the scaffold structure, and from electrospun silk fibroin mats layered with the antibiotic. Mean±SD.

FIG. 2 depicts cumulative in vitro release of cefazolin in 100 μl of water from silk fibroin scaffolds embedded with antibiotic loaded microspheres, layered with antibiotic, embedded with antibiotic directly in the scaffold structure, and from electrospun silk fibroin mats layered with the antibiotic. Mean±SD.

FIGS. 3 A&B depicts cumulative in vitro release of gentamicin (in panel A); and cefazolin (in panel B), in combination in 100 μl of water from silk fibroin scaffolds embedded with antibiotic-loaded microspheres; layered with antibiotics; embedded with antibiotics directly in the scaffold structure; or from electrospun silk fibroin mats layered with the antibiotics. Mean±SD.

FIG. 4 depicts mean zones of clearance of Escherichia coli ATCC 25922 around gentamicin-loaded silk fibroin scaffolds embedded with loaded microspheres; layered with antibiotic; embedded with antibiotic in the scaffold structure; or electrospun silk mats layered with the antibiotic; on Mueller-Hinton agar plates after 24 hr at 37° C. Controls included clearance by a 10 μg gentamicin SENSI-DISC™ antibiotic disc (Becton Dickenson) and an antibiotic-deficient scaffold. Mean±SD.

FIG. 5 depicts mean zones of clearance of Staphylococcus aureus ATCC 25923 around gentamicin-loaded silk fibroin scaffolds embedded with loaded microspheres; layered with antibiotic; embedded with antibiotic in the scaffold structure; or electrospun silk mats layered with the antibiotic, on Mueller-Hinton agar plates after 24 hr at 37° C. Controls included clearance by a 10 μg gentamicin SENSI-DISC™ disc and an antibiotic-deficient scaffold. Mean±SD.

FIG. 6 depicts mean zones of clearance of E. coli ATCC 25922 around cefazolin-loaded silk fibroin scaffolds embedded with loaded microspheres; layered with antibiotic; embedded with antibiotic in the scaffold structure; or electrospun silk mats layered with the antibiotic, on Mueller-Hinton agar plates after 24 hr at 37° C. Controls included clearance by a 10 μg cefazolin SENSI-DISC™ antibiotic disc and an antibiotic-deficient scaffold. Mean±SD.

FIG. 7 depicts mean zones of clearance of S. aureus ATCC 25923 around cefazolin-loaded silk fibroin scaffolds embedded with loaded microspheres; layered with antibiotic; embedded with antibiotic in the scaffold structure; or electrospun silk mats layered with the antibiotic, on Mueller-Hinton agar plates after 24 hr at 37° C. Controls included clearance by a 30 μg cefazolin SENSI-DISC™ disc and an antibiotic-deficient scaffold. Mean±SD.

FIG. 8 depicts mean zones of clearance of E. coli ATCC 25922 around gentamicin/cefazolin-loaded silk fibroin scaffolds embedded with loaded microspheres; layered with antibiotic; embedded with antibiotic in the scaffold structure; or electrospun silk mats layered with the antibiotics, on Mueller-Hinton agar plates after 24 hr at 37° C. Controls included clearance by a 10 μg gentamicin/30 μg cefazolin SENSI-DISC™ disc and an antibiotic-deficient scaffold. Summation of right-set Y-axes represents estimated total antibiotic release. Right set Y-axes cannot be applied to SENSI-DISC™ antibiotic discs. Mean±SD.

FIG. 9 depicts mean zones of clearance of S. aureus ATCC 25923 around gentamicin/cefazolin-loaded silk fibroin scaffolds embedded with loaded microspheres; layered with antibiotic; embedded with antibiotic in the scaffold structure; or electrospun silk mats layered with the antibiotics, on Mueller-Hinton agar plates after 24 hr at 37° C. Controls included clearance by a 10 μg gentamicin/30 μg cefazolin SENSI-DISC™ disc and an antibiotic-deficient scaffold. Summation of right-set Y-axes represents estimated total antibiotic release. Right set Y-axes cannot be applied to SENSI-DISC™ antibiotic discs. Mean±SD.

FIG. 10 shows the optical density of S. aureus and E. coli liquid cultures at 600 nm relative to the concentration of penicillin used in the preparation of antibiotic-containing silk film scaffolds.

FIG. 11 shows S. aureus inhibition from penicillin loaded silk gels over 4 days, prepared from either 4% or 8% (w/v) silk solution either bulk loaded with penicillin by mixing penicillin into the silk solution prior to sonication (bulk) or loaded with microspheres by mixing penicillin silk microspheres into the silk solution prior to sonication (spheres).

FIG. 12 presents S. aureus inhibition from ampicillin loaded 8% (w/v) silk gels over 4 days Silk hydrogels are either bulk loaded with ampicillin by mixing ampicillin into the silk solution prior to gelling (bulk loading) or loaded with microspheres by mixing ampicillin silk microspheres into the silk solution just after sonication.

FIG. 13 demonstrates stability over 140 days (5 months) of penicillin stored in solution or in 8% (w/v) silk films at 4° C. (refrigeration), 25° C. (room temperature), and 37° C. (body temperature). N=3, error bars represent standard deviations.

FIG. 14 shows a comparison of stability for penicillin stored in 8% (w/v) silk films, in solution and as dry powder at 4° C. (refrigeration), 25° C. (room temperature), and 37° C. (body temperature). N=3, error bars represent standard deviations. All x-axis are in days.

FIG. 15 presents a comparison of stability for penicillin stored in 8% (w/v) silk films against 8% (w/v) collagen films at 4° C. (refrigeration), 25° C. (room temperature), and 37° C. (body temperature). N=3, error bars represent standard deviations.

FIG. 16 shows the cumulative release of gentamicin from nanofilm-coated porous silk scaffolds on S. aureus and E. coli lawns (note the close agreement between the gentamicin values determined for the two different bacteria).

FIG. 17 shows the cumulative release of cefazolin from nanofilm coated porous silk scaffolds on S. aureus lawns.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present invention provides for a natural polymeric medium, based on silk fibroin, that comprises a three-dimensional (3-D) silk fibroin scaffold-based formulation of silk protein and at least one antibiotic agent. Such biomaterial compositions offer a unique, medically-relevant, biocompatible structure for medical implants, tissue engineering such as tissue repair, or drug delivery systems to prevent and/or treat microbial contamination. The silk-based compositions of the invention may be formulated to deliver the at least one antibiotic agent at levels exceeding the minimum inhibitory concentration for organisms commonly found to be the cause of such microbial contamination. Various methods may be employed to embed (i.e., incorporate, absorb, or load) at least one antibiotic agent into the silk fibroin scaffolds. For example, the antibiotic may be incorporated directly into silk scaffold by mixing the antibiotic with the silk fibroin solution before scaffold formation; the agent may be loaded in silk microspheres which are then embedded into silk scaffolds (such as porous matrices or gels); or one or more antibiotic-loaded layers are coated on to silk scaffolds. In particular embodiments, silk fibroin microspheres are prepared in the presence of antibiotic to form antibiotic-loaded microspheres, which are then mixed with silk fibroin solution that is formed into a scaffold (e.g., a porous matrix or gel), resulting in antibiotic-loaded microspheres embedded in the silk fibroin scaffold. In other embodiments, the compositions are a coating on a bandage or an implant.

The compositions of the present invention may be used to prevent or treat various microbial contaminations, particularly those caused by surgical site infections. In one embodiment, the common surgical prophylactics such as cefazolin, gentamicin, penicillin, ampicillin, or a combination thereof, may be incorporated into the silk fibroin scaffold to prevent or treat surgical site infections. For example, the drug-embedded silk fibroin scaffolds of the present invention are evaluated for drug release and bacterial clearance of gram-negative E. coli and gram-positive S. aureus, two of the most prevalent pathogens isolated from surgical site infections according to the Centers for Disease Control. The present invention relates to the pharmaceutical utility of silk fibroin scaffolds embedded with antibiotics in providing effective local concentrations of antimicrobial for an appropriate duration of time. Furthermore, silk fibroin based drug delivery or medical implants represents a good medical substitute to systemic prophylaxis for surgery. When combined with the versatile material formats achievable with silk, the mechanically robust nature of these materials, the biocompatibility of this protein, and its controllable proteolytic degradation, antibiotic-functionalized silk biomaterials are an intriguing option for use for many medical needs, as well as for applications in biofilm control in general.

The 3-D porous silk fibroin-based biomaterials of the present invention offer a supportive medium for tissue engineering. Silk fibroin scaffolds are biocompatible, biodegradable, and biochemically versatile. Silks have been employed for applications in biomedical and biotechnological fields. See Sofia et al., 54 J. Biomed. Mater. Res. 139-48 (2001); Sohn et al., 5 Biomacromol. 751-57 (2004); Um et al., 29 Int. J. Biol. Macromol. 91-97 (2001); Kaplan et al., in ACS SYMPOSIUM SERIES, Vol. 544, 2-16 (McGrath & Kaplan, eds., Birkhauser, Boston, Mass., 1994). Silk is popular because of its availability, the ease of purification (Sofia et al., 2001; Sohn et al., 2004; Um et al., 2001), and its attractive properties. See Kaplan et al., 1994; Kaplan et al., PROTEIN BASED MATS. 103-31 (McGrath & Kaplan, eds., Birkhauser, Boston, Mass., 1998); Wang et al., 27 Biomats. 6064-82 (2006).

Silk has an unusual amino acid sequence: the bulk of the silk fibroin protein is organized into hydrophobic domains that are rich of alanine and glycine residues, and amino acids with large side chains that are clustered in chain-end hydrophilic blocks. See Bini et al., 335 J. Mol. Biol. 27-40 (2004). Structurally, the hydrophobic blocks assemble into crystalline regions while the hydrophilic blocks form less ordered regions. Zhou et al., 44 Proteins: Struct. Funct. Bioinf. 119-22 (2001). The large hydrophobic regions of silk fibroin are capable of assembling into crystalline β-sheet structures via intra- and inter-molecular hydrogen bonding and hydrophobic interactions, thus conferring unique features to the silk fibroin protein.

Silk fibroin-based materials promote cellular migration and adherence, the formation of new extracellular matrix, and foster the transport of metabolic wastes and nutrients. Kim et al., 26 Biomats. 2775-85 (2005); Hofmann et al., 111 J. Contr. Rel. 219-27 (2006); Kluge et al., 26 Trends Biotechnol. 244-51 (2008). Silkworm silk from Bombyx mori is composed of the structural protein fibroin and water-soluable glue-like sericins that bind the fibroin fibers together. Magoshi et al., Silk fiber formation, multiple spinning mechanisms, in POLYMERIC MATS. ENCYCLOP. (Salamone, ed., CRC Press, NY, 1996). Fibroin primarily consists of the amino acids glycine, alanine and serine, which form antiparallel, crystalline β-sheet stacks by hydrogen bonding and hydrophobic interactions, forming the basis for the mechanical stability, tensile strength, and toughness of the silk material. Altman et al., 23 Biomats. 4131-41 (2002); Altman et al., 24 Biomats. 401-16 (2003); Kim et al., 2005.

Silk fibroin has been explored as a biomaterial for cardiovascular and musculoskeletal implants, substrates for tissue engineered cartilage, bone, and ligaments; and also in directing the appropriate differentiation of human mesenchymal stem cells into specific tissues. Meinel et al., 88 Biotechnol. Bioeng. 379-91 (2004); Meinel et al. 37 Bone 688-98 (2005); Hofmann et al., 2006. Silk scaffolds provide a viable medium for cellular proliferation and drug delivery, supplying signals such as growth factors and cytokines through protein-release to guide mesenchymal stem cell differentiation. Meinel et al., 2004; Meinel et al., 37 Bone 688-98 (2005); Hofmann et al., 2006. Many synthetic metallic implants and polymers do not degrade under biologically-relevant conditions, and biodegradable synthetic polymers (e.g. polyglycolic acid, poly-L-lactic acid, polyhydroxy-alkanoates, and polyethylene glycol) rapidly lose mechanical strength and fail to foster the production of stable extracellular matrix. Wake et al., 5 Cell Transplant. 465-73 (1996); Kim et al., 251 Exp. Cell. Res. 318-28 (1999); Zhang et al., 29 Biomats. 2217-27 (2008). The utilization of silk in tissue engineering can produce a functional and mechanically effective implant material, stabilizing and releasing bioactive proteins for control of appropriate cellular differentiation and/or growth through controlled drug delivery. Hofmann et al., 2006; Wang et al., 29 Biomats. 894-903 (2008).

In evaluating the efficacy of engineered silk fibroin scaffolds for use in implant or regenerative medicine, a major limitation concerns the susceptibility of the application to microbial contamination. Surgical site infections are the second most common cause of nosocomial infections. Burke 348 N. Engl. J. Med. 651-56 (2003); Bratzler & Houck, 38 Clin. Infect. Dis. 1706-15 (2004). Patients that develop surgical site infections are 60% more likely to be admitted to an intensive care unit, five times more likely to be readmitted to the hospital, and twice more likely to die than patients without surgical site infection. Bratzler & Houck, 2004. Furthermore, patient healthcare costs significantly increase with the incidence of surgical site infection. Kirkland et al., 20 Infect. Control. Hosp. Epidemiol. 725-30 (1999); Hollenbeak et al., 23 Infect. Contr. Hosp. Epidemiol. 177-82 (2002). The National Nosocomial Infections Surveillance (NNIS) system, authorized by the U.S. Centers for Disease Control and Prevention (CDC), established that the distribution of pathogens isolated from surgical site infections has remained constant during the last decade. S. aureus, coagulase-negative staphylococci, Enterococcus species and E. coli are the most frequently isolated pathogens from surgical site infections. NNIS, 27 Am. J. Infect. Contr. 520-32 (1999).

A pervasive and effective method of preventing microbial contamination following surgery is antimicrobial prophylaxis. According to the Surgical Infection Prevention Guideline Writers Workgroup from the Centers for Medicare and Medicaid Services and the CDC, appropriate prophylactics for gynecologic, obstetrical, abdominal, orthopedic, cardiothoracic, vascular, and colorectal surgery often include the first generation cephalosporin antibiotic cefazolin and the aminoglycoside antibiotic gentamicin. Optimal prophylaxis warrants adequate concentrations of appropriate antimicrobials in the serum, tissue, and wound, during surgery and periods of high risk for bacterial contamination. Bratzler & Houck, 2004. There are numerous inconsistencies, however, regarding the appropriate selection, timing, and duration of administration of prophylactic antimicrobials. Id.; Mangram et al., 27 Am. J. Infect. Contr. 132-34 (1999). Additionally, the antimicrobial agent should be administered as near to the incision or implant area as possible to achieve the lowest surgical site infection rates. Classen et al., 326 N. Engl. J. Med. 281-86 (1992); Burke, 348 N. Engl. J. Med. 651-6 (2003); Bratzler & Houck, 2004. Moreover, a systemic antimicrobial approach to infection prevention often results in insufficient local concentrations of antibiotic and significantly increases the risk for surgical site infection. Park et al., 25 Biomats. 3689-98 (2004); Bratzler & Houck, 2004.

One embodiment of the invention relates to a composition comprising a 3-D silk fibroin scaffold-based formulation of silk protein and at least one antibiotic agent. The biomaterial of the invention may be used for medical implants, tissue engineering, regenerative medicine, or drug delivery systems to prevent and/or treat microbial contamination. The composition may be formulated to deliver the at least one antibiotic agent at levels exceeding the MIC for organisms commonly found to be the cause of such microbial contamination. The MIC for a particular antimicrobial agent and a particular microorganism is defined as the minimum concentration of that antimicrobial agent that must be present in an otherwise suitable growth medium for that microorganism, in order to render the growth medium unsuitable for that microorganism, i.e., the minimum concentration to inhibit growth of that microorganism.

As used herein, the term “fibroin” includes silkworm fibroin and insect or spider silk protein. See e.g., Lucas et al., 13 Adv. Protein Chem. 107-242 (1958). Silk fibroin may be obtained from a solution containing a dissolved silkworm silk or spider silk. The silkworm silk protein is obtained, for example, from Bombyx mori, and the spider silk is obtained from Nephila clavipes. In the alternative, the silk proteins suitable for use in the present invention can be obtained from a solution containing a genetically engineered silk, such as from bacteria, yeast, mammalian cells, transgenic animals, or transgenic plants. See, e.g., WO 97/08315; U.S. Pat. No. 5,245,012.

Various methods may be employed to embed at least one antibiotic agent into the silk fibroin scaffolds. In one embodiment, the antibiotic agent(s) is directly incorporated into a silk fibroin scaffold, which may be a 3-D scaffold. In another embodiment, the antibiotic agent(s) is mixed with silk fibroin solution, then a silk fibroin scaffold is coated with one or more antibiotic agent(s)-loaded layers by dipping the silk fibroin scaffold in an antibiotic-loaded silk fibroin solution and drying the resulting structure. In another embodiment, the steps of preparing the antibiotic-containing composition comprise preparing silk microspheres that incorporate at least one antibiotic agent; mixing the antibiotic-loaded silk microspheres with a silk fibroin aqueous salt solution; and removing the salt and water from the solution to form a 3-D silk fibroin scaffold embedded with the antibiotic-loaded silk microspheres. In yet another embodiment, the steps of preparing the biomaterial comprise preparing silk microspheres loaded with at least one antibiotic agent; mixing the antibiotic-loaded silk microspheres with a silk fibroin solution; and sonicating the solution to form a 3-D silk fibroin gel scaffold embedded with antibiotic-loaded silk microspheres. In another embodiment, the antibiotic-containing silk composition is used as a coating on a substrate, such as a bandage or implant.

The present invention also encompasses other methods of embedding antibiotic agents into the silk fibroin scaffolds commonly used in drug delivery. Silk fibroin matrix may be prepared from an aqueous silk fibroin solution, which may be prepared from the silkworm cocoons using techniques known in the art. See, e.g., U.S. patent application Ser. No. 11/247,358; WO/2005/012606; WO/2008/127401. The silk aqueous solution can then be processed into silk fibroin matrices using a variety of processing techniques, such as electrospinning (Jin et al., 3 Biomacromol. 1233-39 (2002)), sonication (Wang et al., 29 Biomats. 1054-64 (2008)), or chemical modification through covalent binding (Murphy et al., 29 Biomats. 2829-38 (2008)). These processes yield silk biomaterials that are formed and/or stabilized through β-sheet assembly, with the mechanical properties and enzymatic degradation rates of silks depending on the size and distribution of these crystalline β-sheet regions. See, e.g., Asakura et al., 42 Magn. Reson. Chem. 258-66 (2004). For example, the silk scaffold may comprise a porous silk fibroin material made by freeze-drying, salt leaching or gas foaming. See WO 2004/062697.

Antibiotic agents that can be embedded to the biomaterials of the present invention include, but are not limited to, actinomycin; aminoglycosides (e.g., neomycin, gentamicin, tobramycin); beta-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above. In one embodiment of the invention, the antibiotic agent is cefazolin, gentamicin, or a combination thereof.

Additionally, the antibiotic-loaded scaffolds of the present invention may comprise other components such as at least one active agent. The agent may be embedded in the scaffold or immobilized on the scaffold. More specifically, embedding an additional active agent in the composition may be achieved by introducing the active agent to silk fibroin-based solutions prior to or when mixing the antibiotic. Alternatively, the active agent may be introduced to the silk fibroin-based composition after the formation of the antibiotic-containing scaffold structure.

The variety of active agents that can be used in conjunction with the silk fibroin-based scaffolds of the present invention is vast. For example, the active agent may be a therapeutic agent or biological material, such as cells, proteins, peptides, nucleic acid analogues, nucleotides, oligonucleotides, nucleic acids (DNA, RNA, siRNA), peptide nucleic acids, aptamers, antibodies or fragments or portions thereof, antigens or epitopes, hormones, hormone antagonists, growth factors or recombinant growth factors and fragments and variants thereof, cell attachment mediators (such as RGD), cytokines, enzymes, anti-inflammation agent, antifungals, antivirals, toxins, prodrugs, chemotherapeutic agents, small molecules, drugs (e.g., drugs, dyes, amino acids, vitamins, antioxidants), other antimicrobial compounds, and combinations thereof. See, e.g., PCT/US09/44117; U.S. Patent Application Ser. No. 61/224,618.

In some embodiments, the active agent may also be an organism such as a fungus, plant or animal, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

Exemplary cells suitable for use herein may include, but are not limited to, progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. See also WO 2008/106485; PCT/US2009/059547; WO 2007/103442.

Exemplary antibodies include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

Additional active agents include cell growth media, such as Dulbecco's Modified Eagle Medium, fetal bovine serum, non-essential amino acids and antibiotics; growth and morphogenic factors such as fibroblast growth factor, transforming growth factors, vascular endothelial growth factor, epidermal growth factor, platelet derived growth factor, insulin-like growth factors), bone morphogenetic growth factors, bone morphogenetic-like proteins, transforming growth factors, nerve growth factors, and related proteins (growth factors are known in the art, see, e.g., Rosen & Thies, CELLULAR & MOLECULAR BASIS BONE FORMATION & REPAIR (R.G. Landes Co.); anti-angiogenic proteins such as endostatin, and other naturally derived or genetically engineered proteins; polysaccharides, glycoproteins, or lipoproteins; anti-infectives such as antibiotics and antiviral agents, chemotherapeutic agents (i.e., anticancer agents), anti-rejection agents, analgesics and analgesic combinations, anti-inflammatory agents, and steroids.

Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like. Interactions between components may also be used to functionalize silk fibroin through, for example, specific interaction between avidin and biotin. See U.S. Patent Application Ser. No. Ser. No. 61/226,801.

The embodiments of the present invention may also include suitable biocompatible material in the silk fibroin scaffolds, such as polyethylene oxide (see, e.g., U.S. Patent Application Ser. No. 61/225,335), polyethylene glycol (see PCT/US09/64673), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, glycerol (see PCT/US2009/060135), and other biocompatible polymers, see WO 2004/0000915. Additionally, some or all of the silk scaffold may be coated with an inorganic material by forming an anionic polymer interface on the silk fibroin and contacting the interface with a mineralizing substance, see WO 2005/000483. Alternatively, the silk may be mixed with hydroxyapatite particles, see PCT/US08/82487. As noted herein, the silk fibroin may be of recombinant origin, which provides for further modification of the silk such as the inclusion of a fusion polypeptide comprising a fibrous protein domain and a mineralization domain, which are used to form an organic-inorganic composite. These organic-inorganic composites can be constructed from the nano- to the macro-scale depending on the size of the fibrous protein fusion domain used, see WO 2006/076711. See also U.S. patent application Ser. No. 12/192,588.

Additional silk-based structures may be included in, or otherwise comprise, the antibiotic scaffolds of the present invention. For example, the scaffolds may include grooves (WO 2008/106485); or microchannels (WO 2006/042287; WO 2008/127403; WO 2008/127405); or tubes (WO 2009/023615); or other structure (PCT/US2009/039870); and, optionally, cells within these structured scaffolds, see also WO/2008/108838. The scaffolds of the present invention may comprise an immobilized agent gradient or contain gradient of antibiotic- or agent-loaded microspheres. See, e.g., Wang et al., 134 J. Contr. Release 81-90 (2009). The silk scaffold may be activated in homogenous or gradient fashion using, e.g., carbodiimide chemistry (see U.S. Patent Application Pub. No. 2007/0212730), diazonium coupling reaction (see, e.g., U.S. patent application Ser. No. 12/192,588), or and pegylation with a chemically active or activated derivatives of the PEG polymer (see, e.g., PCT/US09/64673). Additional components or active agents may be loaded layer-by-layer on the silk scaffolds as described herein and, for example, WO 2007/016524. Silk microfluidic scaffolds may be of particular use in wound healing, see PCT/US09/067006.

The silk fibroin scaffolds for antibiotic delivery of the present invention may also comprise an identifying mark such as a photonic imprint (e.g., a hologram) (see PCT/US08/82487; PCT/US09/47751); or be incorporated into or otherwise comprise a silk-based biopolymer optical device having a nanopatterned surface (see WO 2008/127404; WO 2008/118211; WO 2008/127402; WO 2008/140562), biodegradable electronic device (see WO/2008/085904), or reflective surface (see U.S. Patent Application Ser. No. 61/226,801). For example, an antibiotic-containing silk scaffold may be marked with an expiration date and/or manufacturer's label to indicate authenticity. See PCT/US09/47751.

The present invention also provides for compositions and methods for long term storage and stabilizing antibiotics by incorporating them into silk scaffolds. For example, dating back to Fleming's original 1929 paper on penicillin, the literature reports that penicillin is unstable in solution, breaking down within weeks at room temperature (25° C.) and within 24 hr at 37° C. See, e.g., Benedict et al., 49 J. Bacteriol. 85-95 (1945). Breakdown of penicillin at body temperature represents a serious problem for any implantable delivery system designed to release over a time period longer than 24 hours. Additionally, instability of antibiotics at temperatures ≥25° C. represents a problem in transporting and storing antibiotics (particularly in places where refrigeration is limited). Surprisingly, when incorporated in the silk scaffolds of the present invention, penicillin is stable (i.e., maintaining at least 50% of residual activity) for at least 30 days at room temperature (25° C.) and body temperature (37° C.). Hence, temperature-sensitive antibiotics be stored in silk fibroin scaffolds without refrigeration. Importantly, temperature-sensitive antibiotics can be delivered into the body in silk scaffolds and maintain activity for a longer period of time than previously imagined.

The present invention also relates to a method of preventing and/or treating microbial contamination at a region of a subject for medical implants, tissue engineering, or drug delivery. The method comprises contacting said region of the subject with a material including a silk fibroin scaffold comprising at least one antibiotic. The compositions of the invention may be formulated to deliver at least one antibiotic agent at levels exceeding the MIC for an organism commonly found to be the cause of such microbial contamination. Thus, for example, the antibiotic-containing scaffold has a therapeutic or prophylactic effect (as well as agents that have positive pharmacological effects) on the expression of the extracellular matrix. In this regard, for example the bioactive agent can enhance wound healing (e.g., at a vascular site).

Indeed, the antibiotic scaffolds of the present invention may be used in a variety of medical applications, such as a drug (e.g., small molecule, protein, or nucleic acid) delivery device, including controlled release systems, wound closure systems, including vascular wound repair devices, hemostatic dressings, bandages, patches and glues, sutures, and in tissue engineering applications, such as, for example, scaffolds for tissue regeneration, ligament prosthetic devices and in products for long-term or bio-degradable implantation into an animal or human body.

Controlled release of the antibiotic and/or additional active agent from the silk composition may be designed to occur over time, for example, for greater than about 12 hour or 24 hour, inclusive; greater than one month or two months or five months, inclusive. The time of release may be selected, for example, to occur over a time period of about 12 hour to 24 hour, or about 12 hour to 1 week. In another embodiment, release may occur for example on the order of about 1 month to 2 months, inclusive. The controlled release time may be selected based on the condition treated. For example, a particular release profile may be more effective for wound healing or where consistent release and high local dosage are desired.

Methods of prevention and/or treatment of microbial contamination, particularly those caused by surgical site infection are encompassed by the present invention. Surgical site infections are one of the most common causes of nosocomial infections and represent an enormous problem for patient safety and public health. Surgical site infections that may be treated or prevented by using the biomaterials of the present invention include, but not limited to, the bacterial infections such as Streptococcus pyogenes (S. pyogenes), Pseudomonas aeruginosa (P. aeruginosa), Enterococcus faecalis (E. faecalis), Proteus mirabilis (P. mirabilis), Serratia marcescens (S. marcescens), Enterobacter clocae (E. clocae), Acetinobacter anitratus (A. anitratus), Klebsiella pneumoniae (K. pneumonia), E. coli, S. aureus, coagulase-negative Staphylococci, and Enterococcus spp, and so forth. The methods of the invention are effective for any surgical site infection including, but not limited to, gynecologic, obstetrical, abdominal, orthopedic, cardiothoracic, vascular, and colorectal surgeries. The target regions of a mammalian body, in particular human, for preventing or treating microbial contamination include, but not limited to, regions such as skin, lung, bone, joint, stomach, blood, heart valve, urinary tract or other regions that may have microbial contaminations, or may be particularly prone to surgical site infections.

The formulation can be administered to a patient in need of the antibiotic that has been encapsulated in the composition. The pharmaceutical formulation may be administered by a variety of routes known in the art including topical, oral, ocular, nasal, transdermal or parenteral (including intravenous, intraperitoneal, intramuscular and subcutaneous injection as well as intranasal or inhalation administration) and implantation. The delivery may be regional or local. Additionally, the delivery may be intrathecal, e.g., for CNS delivery.

When desired, the antibiotic-containing silk scaffold may include a targeting ligand or precursor targeting ligand. Targeting ligand refers to any material or substance which may promote targeting of a pharmaceutical formulation to tissues and/or receptors in vivo and/or in vitro. The targeting ligand may be synthetic, semi-synthetic, or naturally-occurring. Materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steroids, steroid analogs, hormones, cofactors, and genetic material, including nucleosides, nucleotides, nucleotide acid constructs, peptide nucleic acids (PNA), aptamers, and polynucleotides. Other targeting ligands in the present invention include cell adhesion molecules (CAM), among which are, for example, cytokines, integrins, cadherins, immunoglobulins and selectin. A precursor to a targeting ligand refers to any material or substance which may be converted to a targeting ligand. Such conversion may involve, for example, anchoring a precursor to a targeting ligand. Exemplary targeting precursor moieties include maleimide groups, disulfide groups, such as ortho-pyridyl disulfide, vinylsulfone groups, azide groups, and iodo acetyl groups.

In preparation for in vivo application, the silk-based scaffolds of the present invention may be formulated to include excipients. Exemplary excipients include diluents, solvents, buffers, or other liquid vehicle, solubilizers, dispersing or suspending agents, isotonic agents, viscosity controlling agents, binders, lubricants, surfactants, preservatives, stabilizers and the like, as suited to particular dosage form desired. The formulations may also include bulking agents, chelating agents, and antioxidants. Where parenteral formulations are used, the formulation may additionally or alternately include sugars, amino acids, or electrolytes.

More specifically, examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; oils such as peanut oil, cottonseed oil; safflower oil, sesame oil; olive oil; corn oil and soybean oil; esters such as ethyl oleate and ethyl laurate; agar; non-toxic compatible lubricants such as sodium lauryl sulfate and magnesium stearate; polyols, for example, of a molecular weight less than about 70,000 kD, such as trehalose, mannitol, and polyethylene glycol. See, e.g., U.S. Pat. No. 5,589,167. Exemplary surfactants include nonionic surfactants, such as Tween surfactants, polysorbates, such as polysorbate 20 or 80, etc., and the poloxamers, such as poloxamer 184 or 188, pluronic polyols, and other ethylene/polypropylene block polymers, etc. Suitable buffers include Tris, citrate, succinate, acetate, or histidine buffers. Suitable preservatives include phenol, benzyl alcohol, metacresol, methyl paraben, propyl paraben, benzalconium chloride, and benzethonium chloride. Other additives include carboxymethylcellulose, dextran, and gelatin. Suitable stabilizing agents include heparin, pentosan polysulfate and other heparinoids, and divalent cations such as magnesium and zinc. Coloring agents, releasing agents, coating agents, sweetening, flavoring and perfuming agents, preservatives and antioxidants can also be present in the composition, according to the judgment of the formulator.

Some embodiments of the present invention relate to the utility of silk fibroin based biomaterials as antibiotic drug delivery systems for potential utility in medical implants, tissue repairs and for medical device coatings. In particular, the common surgical prophylactic antibiotics such as cefazolin and gentamicin, or a combination thereof, are embedded into silk scaffolds using a variety of methods. Drug embedded silk scaffolds may be evaluated for drug release kinetics and bacterial clearance of, for example, E. coli and S. aureus, prevalent pathogens isolated from surgical site infections.

The primary objective of antimicrobial drug therapy is to maximize the therapeutic benefits while minimizing adverse side-effects such as bacterial resistance and toxicity. Domb et al., 3 Polym. Adv. Technol. 279-92 (1993). Drug administration via intravenous or intramuscular injections, oral dosing, and other routes of dispensation, result in a generalized and systemic distribution of the antibiotic to various organs and tissues perfused with blood, with a small, undefined amount reaching the target. Domb et al., (1993); Park et al., 52 J. Contr. Release 179-89 (1998). The present invention provides for natural, biocompatible, and biodegradable polymers such as silk fibroin for application such as medical implants that allow for effective local drug release at controlled rates. This result can possibly eliminate the need for antibacterial prophylaxis and continued drug administration following surgery.

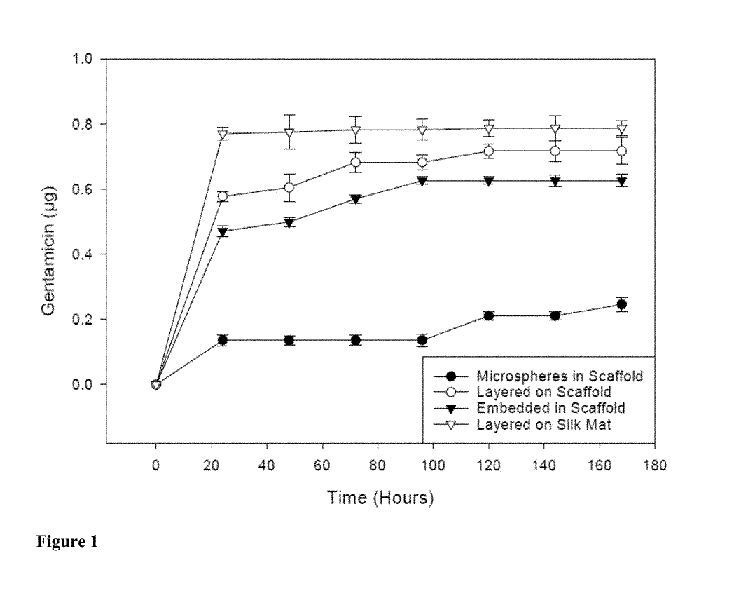

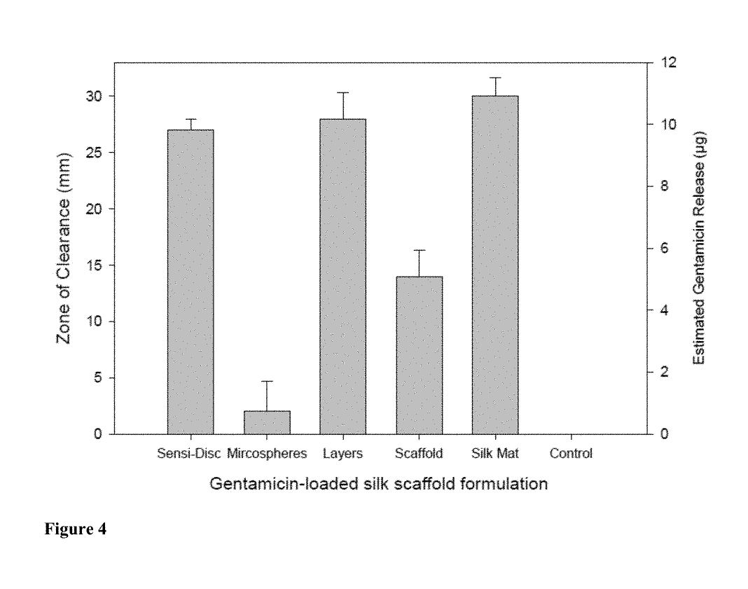

FIGS. 1 to 3 demonstrate the in vitro antibiotic release profiles of gentamicin, cefazolin, and combination of gentamicin and cefazolin from silk fibroin based scaffold embedded with these antibiotics through different methods, including embedding the antibiotic agent(s) directly into silk scaffold, embedding the antibiotic loaded-silk microspheres into silk scaffolds, coating the silk scaffold with one or more antibiotic-loaded layers, or coating the electrospun silk fibroin mats with the antibiotic-loaded layers. FIGS. 4 to 9 demonstrate the in vitro bacterial clearance profiles of E. coli ATCC 25922 and S. aureus ATCC 25923 by using gentamicin, cefazolin, and combination of gentamicin and cefazolin loaded silk materials using the embedding methods discussed herein.

The antibiotic release profiles of gentamicin from silk fibroin structures, as shown in FIG. 1, are different depending on the drug-silk scaffold formulation. All drug loadings displayed an initial burst of gentamicin release within 24 hours followed by a rapid decrease of the release rate to near zero, with the treatment of embedding the gentamicin loaded-silk microspheres into silk scaffolds exhibiting the smallest burst in release. After 24 hours, the spectrophotometrically detected in vitro release profile of gentamicin for the silk scaffolds embedded with gentamicin-loaded silk microspheres (0.136 μg±0.017 μg; p<0.05) was significantly lower than the silk scaffolds layered with gentamicin (0.577 μg±0.016 μg), the silk scaffolds embedded with gentamicin directly in the fibroin structure (0.471 μg±0.017 μg), and the electrospun silk fibroin mats layered with the antibiotic (0.770 μg±0.020 μg).

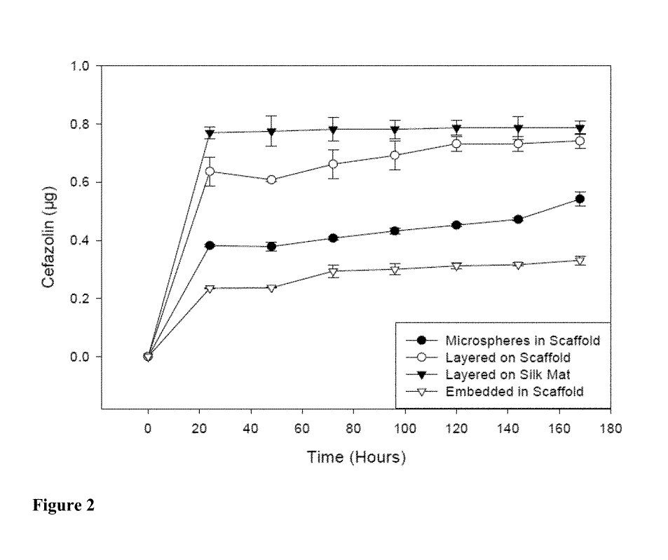

Spectrophotometrically detected in vitro antibiotic release profiles of cefazolin, as shown in FIG. 2, are different depending on the drug-silk scaffold formulation. As was observed with the gentamicin-release profiles, all drug loadings displayed an initial burst of antibiotic release within 24 hours followed by a sharp decrease in the release rate. Coating the silk scaffold with agent-loaded layers yielded a rate of release that became essentially zero after the 24 hour burst. In contrast, antibiotics embedded directly in scaffolds or entrapped in silk microspheres in the scaffolds exhibited the smallest burst in release, but continued to release drug at a low rate throughout the testing period. The cefazolin release after 24 hours was lower for the formulations of cefazolin embedded directly into silk scaffolds (0.235 μg±0.001 μg; p<0.05) and cefazolin-loaded silk microspheres embedded into silk scaffold (0.382 μg±0.005 μg; p<0.05), than for the formulations of silk scaffolds layered with cefazolin (0.637 μg±0.050 μg) and the electrospun silk fibroin mats layered with antibiotic (0.770 μg±0.019 μg).

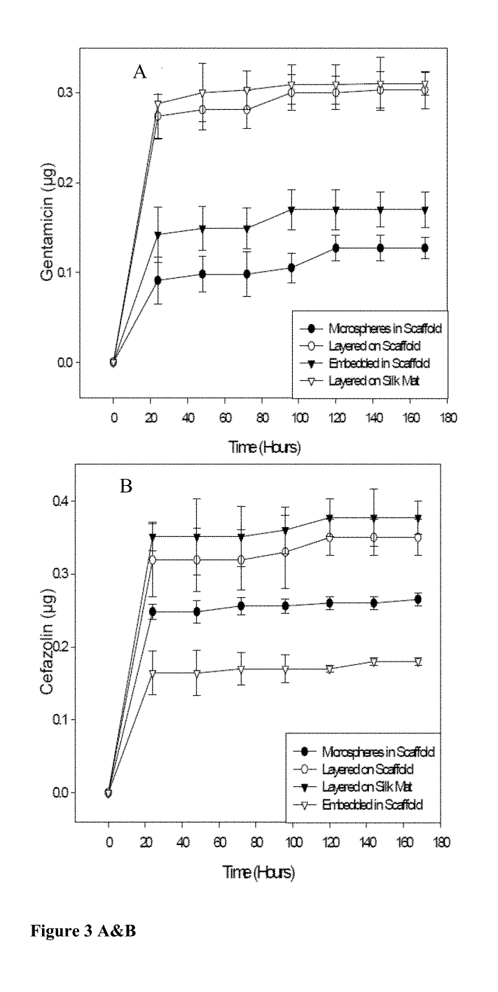

The in vitro antibiotic release profiles of gentamicin/cefazolin combination, as shown in FIG. 3, are different depending on the drug-silk scaffold formulation. All drug loadings displayed an initial burst of antibiotic release within 24 hours followed by a leveling-off of the release rate. Spectrophotometrically detected antibiotic release after 24 hours was significantly lower from silk scaffolds embedded with gentamicin/cefazolin-loaded silk microspheres (0.136 μg±0.017 μg; p<0.05) than scaffolds layered with gentamicin/cefazolin (0.577 μg±0.016 μg), scaffolds with gentamicin/cefazolin embedded directly in the fibroin structure (0.471 μg±0.017 μg), and electrospun fibroin mats layered with the antibiotics (0.770 μg±0.020 μg).

Disc diffusion of gentamicin represented as mean zones of clearance of E. coli ATCC 25922, as shown in FIG. 4, after 24 hours was significantly lower from silk scaffolds with gentamicin embedded directly in the silk fibroin structure (14.0 mm±2.30 mm; p<0.05) and from silk scaffolds embedded with gentamicin-loaded silk microspheres (2.00 mm±2.67 mm; p<0.05) than all other drug-silk formulations. Scaffolds layered with gentamicin (28.0 mm±2.33 mm) and electrospun fibroin mats layered with the antibiotics (30.0 mm±1.67 mm) cleared E. coli with a zone similar to the 10 μg gentamicin SENSI-DISC™ disc (27.0 mm±1.00 mm). SENSI-DISC™ antibiotic disc zone of clearance values did not statistically differ from values established by the NCCLS Document M100-S13 (M2): Disc Diffusion Supplemental Tables (NCCLS, Wayne, Pa., 2003) (hereinafter NCCLS, 2003), verifying the validity of the present results (p>0.05). Based on zones of clearance by SENSI-DISC™ antibiotic disc controls, gentamicin release for each drug-silk formulation was estimated (FIG. 4).

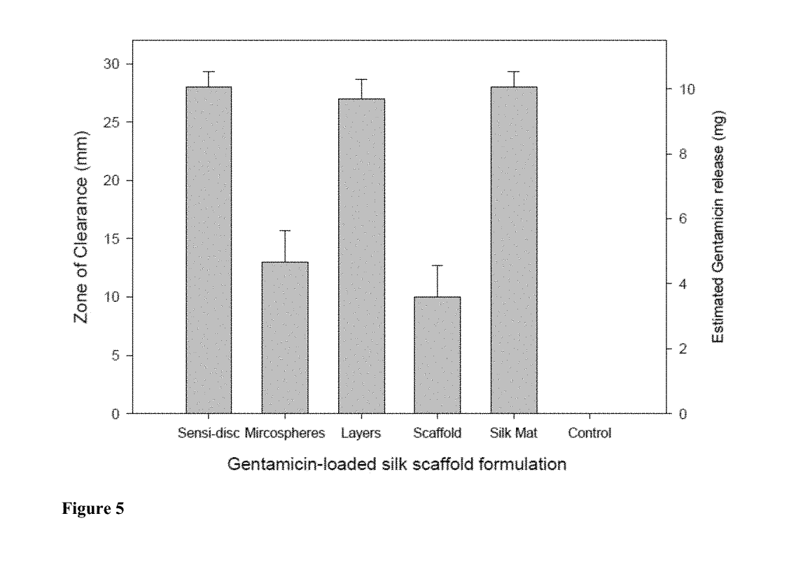

Disc diffusion of gentamicin represented as mean zones of clearance of S. aureus ATCC 25923, as shown in FIG. 5, after 24 hours was lower from silk scaffolds embedded with gentamicin directly in the silk fibroin structure (10.0 mm±2.67 mm; p<0.05) and from silk scaffolds embedded with gentamicin-loaded silk microspheres (13.0 mm±2.67 mm; p<0.05) than all other drug-silk formulations. Scaffolds layered with gentamicin (27.0 mm±2.33 mm) and electrospun fibroin mats layered with the antibiotic (28.0 mm±1.33 mm) cleared S. aureus similar to the 10 μg gentamicin SENSI-DISC™ disc (28.0 mm±1.33 mm). Zone of clearance values obtained from SENSI-DISC™ antibiotic disc diffusion did not statistically differ from values established by NCCLS, 2003, verifying the validity of the present results (p>0.05). Based on zones of clearance by SENSI-DISC™ antibiotic disc controls, gentamicin release for each drug-silk formulation was estimated (FIG. 5).

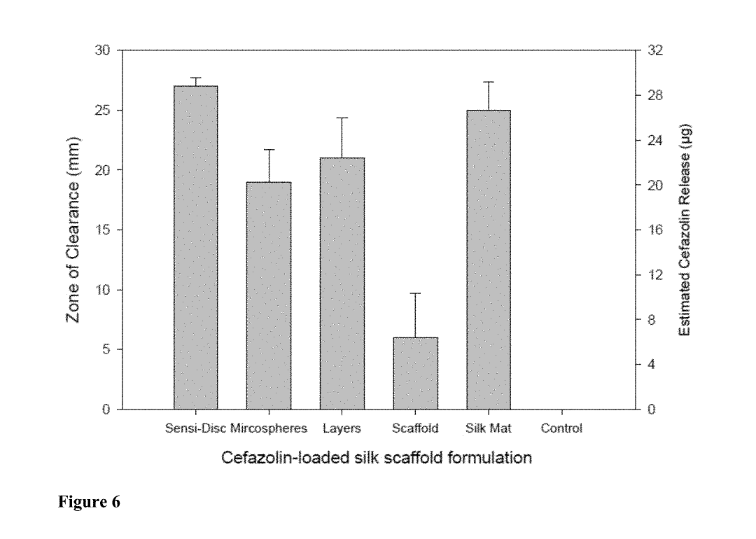

Disc diffusion of cefazolin represented as mean zones of clearance of E. coli ATCC 25922, as shown in FIG. 6, after 24 hours was significantly lower from silk scaffolds embedded with cefazolin directly in the silk fibroin structure (6.00 mm±3.67 mm; p<0.05) than all other drug-silk formulations. Silk scaffolds embedded with cefazolin-loaded silk microspheres (19.0 mm±2.67 mm), layered with cefazolin (21.0 mm±3.33 mm), and electrospun fibroin mats layered with the antibiotic (25.0 mm±2.33 mm) cleared E. coli similar to the 30 μg cefazolin SENSI-DISC™ disc (27.0 mm±0.67 mm). SENSI-DISC™ antibiotic disc zone of clearance values did not statistically differ from values established by the NCCLS, 2003, verifying the validity of the present results (p>0.05). Based on zones of clearance by SENSI-DISC™ antibiotic disc controls, gentamicin release for each drug-silk formulation was estimated (FIG. 6).

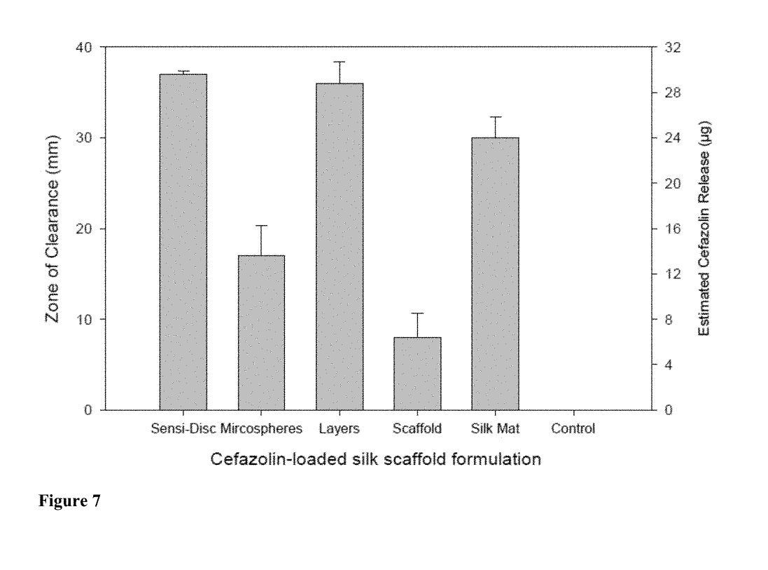

Disc diffusion of cefazolin represented as mean zones of clearance of S. aureus ATCC 25923 after 24 hours, as shown in FIG. 7, was significantly lower from silk scaffolds embedded with cefazolin directly in the silk fibroin structure (8.00 mm±2.67 mm; p<0.05) than all other drug-silk formulations. Silk scaffolds embedded with cefazolin-loaded silk microspheres (17.0 mm±3.33 mm), layered with cefazolin (36.0 mm±2.33 mm), and electrospun fibroin mats layered with the antibiotic (30.0 mm±2.33 mm) cleared S. aureus similar to the 30 μg cefazolin SENSI-DISC™ disc (37.0 mm±0.33 mm). SENSI-DISC™ antibiotic disc zone of clearance values did not statistically differ from values established by the NCCLS, 2003, verifying the validity of the present results (p>0.05). Based on zones of clearance by SENSI-DISC™ antibiotic disc controls, cefazolin release for each drug-silk formulation was estimated (FIG. 7).

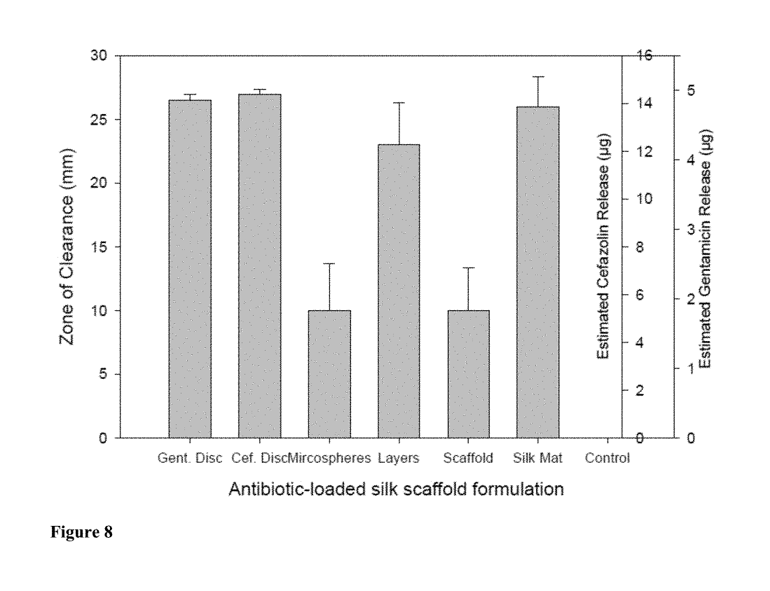

Disc diffusion of gentamicin/cefazolin represented as mean zones of clearance of E. coli ATCC 25922 (FIG. 8), after 24 hours was significantly lower from silk scaffolds embedded with gentamicin/cefazolin directly in the silk fibroin structure (10.00 mm±3.33 mm; p<0.05) and from silk scaffolds embedded with gentamicin/cefazolin-loaded silk microspheres (10.0 mm±3.67 mm; p<0.05) than all other drug-silk formulations. Silk scaffolds layered with gentamicin/cefazolin (23.0 mm±3.33 mm) and electrospun fibroin mats layered with the antibiotics (26.0 mm±2.33 mm) cleared E. coli similar to the 10 μg gentamicin SENSI-DISC™ disc (26.5 mm±0.50 mm) and 30 μg cefazolin SENSI-DISC™ disc (27.0 mm±0.40 mm). Based on zones of clearance by SENSI-DISC™ antibiotic disc controls, gentamicin/cefazolin release for each drug-silk formulation was estimated (e.g., silk scaffolds layered with gentamicin/cefazolin were estimated to simultaneously release 12 μg Cefazolin and 4 μg Gentamicin on a Mueller-Hinton agar plate (FIG. 8).

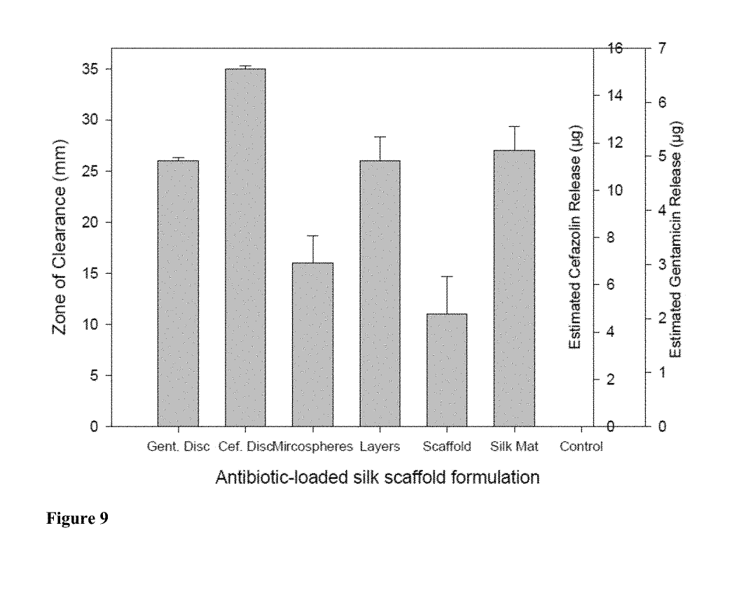

Disc diffusion of gentamicin/cefazolin represented as mean zones of clearance of S. aureus ATCC 25923, after 24 hours was significantly lower from silk scaffolds embedded with gentamicin/cefazolin directly in the silk fibroin structure (11.00 mm±3.67 mm; p<0.05) and from silk scaffolds embedded with gentamicin/cefazolin-loaded silk microspheres (16.0 mm±2.67 mm; p<0.05) than all other drug-silk formulations (FIG. 9). Silk scaffolds layered with gentamicin/cefazolin (26.0 mm±2.33 mm) and electrospun fibroin mats layered with the antibiotic (27.0 mm±2.33 mm) cleared S. aureus similar to the 10 μg gentamicin SENSI-DISC™ disc (26.0 mm±0.34 mm) and 30 μg cefazolin SENSI-DISC™ (35.0 mm±0.31 mm). Based on zones of clearance by SENSI-DISC™ antibiotic disc controls, gentamicin/cefazolin release for each drug-silk formulation was estimated (FIG. 9).

The release profiles of gentamicin and cefazolin formulations when placed in water showed a burst of release within 24 hours, followed by a plateau (FIGS. 1 to 3). Antibiotic release from silk scaffolds may be controlled primarily by diffusion through the polymer matrix, mediated by crystalline β-sheet content and the dissolution properties of the drug. Gentamicin and cefazolin are highly hydrophilic compounds and easily diffused through the porous silk structures and conduits in the scaffolds. Park et al., 1998; Naraharisetti et al., 77 J. Biomed. Mater. Res. B. Appl. Biomater. 329-37 (2006). Advantageously, the antibiotic release trends corresponded with established guidelines for prophylaxis established by the Surgical Infection Prevention Guideline Writers Workgroup from the Centers for Medicare and Medicaid Services and the CDC. The guidelines state that prophylaxis should end within 24 hours following surgery; prolonged use of prophylactic antimicrobials is associated with the dangerous emergence of resistance bacteria. Burke, 348 N. Engl. J. Med. 651-56 (2003); Bratzler & Houck, 2004.

More specifically, the low release rates exhibited by scaffolds embedded with antibiotic-encapsulated silk microspheres may be attributed to their preparation with DOPC lipid vesicles. Although the lipid templates were removed after lyophilization, residual lipid might form an aqueous diffusion boundary layer, providing resistance to the diffusion and dissolution of the antibiotics. Wang et al., 351 Int. J. Pharm. 219-26 (2008); Park et al., 1998. This might also explain the comparably smaller zones of clearance by scaffolds embedded with antibiotic-loaded silk microspheres and scaffolds with the antibiotics embedded directly into the structure. Furthermore, drug-loaded silk microspheres offer applicability in long-term, sustained drug release conditions (Wang et al., 2008). The modest drug release from scaffolds embedded with the antibiotics directly into the silk structure may relate to differences in localized structures in the aqueous silk preparations. Kim et al., 26 Biomats. 2775-85 (2005); Hofmann et al., 2006.

According to the NCCLS (2000), the MIC values established for clearance of E. coli ATCC 25922 and S. aureus ATCC 25923 are 0.5 mg/L and 0.25 mg/L for gentamicin, respectively, and 1.0 mg/L and 0.25 mg/L for cefazolin, respectively. The release of antibiotics in the embodiments of the invention revealed unit adjusted concentrations in the range of 1.0 mg/mL to 10.0 mg/L, exceeding standardized MIC values. To release a lower concentration of antibiotics comparable to the standardized MIC values, a lower initial loading of antibiotics may be embedded into the silk material systems.

Antimicrobial susceptibility testing on Mueller-Hinton agar plates with E. coli ATCC 25922 and S. aureus ATCC 25923 paralleled results from the release experiments, showing a dose-dependent effect as expected (e.g., scaffolds layered with gentamicin or cefazolin released greater amounts of the drug and therefore produced greater zones of clearance; FIGS. 3 to 9). Scaffolds prepared with antibiotic-loaded silk microspheres or with the antibiotics embedded directly into the structure typically displayed smaller zones of clearance than other antibiotic-silk preparations. Generally, silk scaffolds and electrospun mats layered with antibiotic cleared E. coli ATCC 25922 and S. aureus ATCC 25923 similar to standardized SENSI-DISC™ antibiotic discs preloaded with gentamicin or cefazolin. The gentamicin/cefazolin combination did not result in enhanced antibacterial properties.

Based on zones of clearance by standardized SENSI-DISC™ discs embedded with 10 μg gentamicin or 30 μg cefazolin, the amount of the antibiotics encapsulated in each equally sized scaffold was extrapolated (Right set Y-axes, FIGS. 4 to 9). This estimation also describes the antibacterial and drug diffusion properties of each silk-antibiotic preparation. The majority of the antibiotics is released within 24 hours and, overall, the trend in release kinetics does not vary considerably across treatments (FIGS. 1 to 3). For example, the 30 μg cefazolin disc of 6 mm diameter cleared 37.0 mm±0.33 mm of S. aureus ATCC 25923 and the 6 mm diameter scaffold layered with cefazolin cleared 36.0 mm±2.33 mm S. aureus. Therefore, an estimated 29.2 μg of cefazolin was assumed to be encapsulated in the 6 mm scaffold layered with the antibiotic.

The difference between spectrophotometrically detected antibiotic release in water and proportionately calculated antibiotic released on Mueller-Hinton agar plates inoculated with bacteria relates to the varied conditions of each experiment. Drug diffusion is enhanced upon direct contact with agar versus water (Clutterbuck et al., 2007). In addition, SENSI-DISC™ antibiotic discs were assumed to release 10 μg gentamicin or 30 μg cefazolin onto the agar and zones of clearance were equated with the given drug-disc concentration. Smaller concentrations of the drug could have been emitted from the discs, however, proportionately lowering the estimated antibiotic released from the drug-silk preparations.

A lack of consensus exists on the use of antibiotics in peripheral wound healing. Apparently, the antimicrobials tobramicin, gentamicin, and chloramphenicol yielded no beneficial effect on healing rates or quality of healing when topically applied to corneal epithelial wounds in rabbits, and produced fatally toxic systemic effects. Stern et al., 101 Arch. Ophthalmol. 644-47 (1983). Conversely, tobramicin-loaded collagen-hyaluronic acid matrices containing growth factors have significantly enhanced skin wound healing in guinea pigs, and these preparations did not exhibit toxic consequences. Park et al., 2004.

According to the embodiments of the invention, the biomaterials of the invention and the method of using such biomaterials to prevent and/or treat microbial contamination meet the established MIC values for bacterial clearance of E. coli ATCC 25922 and S. aureus ATCC 25923 established by the NCCLS, and thus have potential in pharmaceutical applications for delivering antibiotics. Furthermore, the present invention provides for the effective local concentrations of antimicrobial and appropriate duration of release for the use of silk fibroin polymeric devices and implants as drug delivery systems in tissue repair and for medical devices. Hence, the silk fibroin based biomaterials embedded with antibiotics can potentially offer a new medical substitute to systemic prophylaxis for surgery.

The invention will be further characterized by the following examples which are intended to be exemplary of the embodiments.

### EXAMPLES

### Example 1. Preparation of Silk Fibroin Aqueous Solution

Silk fibroin aqueous stock solutions were prepared as previously described. Hofmann et al., 2006). Briefly, cocoons of B. mori were boiled for 20 min in an aqueous solution of 0.02 M Na₂CO₃, and then rinsed thoroughly with distilled water to extract sericin proteins. The extracted silk fibroin was then dissolved in 9.3 m LiBr solution at 60° C. for 4 hr, yielding a 20% (w/v) solution. This solution was dialyzed against distilled water using a Slide-a-Lyzer dialysis cassette (MWCO 3500 g/mol, Pierce, Woburn, Mass.) at room temperature for 48 hr to remove salts. The dialysate was centrifuged two times, each at 4° C. for 20 min, to remove impurities and the aggregates that formed during dialysis. The final concentration of silk fibroin aqueous solution was approximately 8% (wt/v). Fibroin concentration was determined by weighing the residual solid of a known volume of solution after drying at 60° C. for 24 hr.

If desired, the silk fibroin solution may be further concentrated as taught in WO 2005/012606. As discussed elsewhere herein, the β-sheet content of the silk fibroin may be induced. See, e.g., WO 2005/123114.

### Example 2. Preparation of Antibiotic-Loaded Silk Fibroin Scaffolds

For Preparation of silk fibroin scaffolds, aqueous-derived silk fibroin scaffolds were prepared by the addition of 4 g of granular NaCl₂ (particle size: 600 μm-710 μm) into 2 ml of 6% silk fibroin aqueous solutions in disc-shaped containers. Kim et al., 2005. The container was covered and left at room temperature for 24 hr. The container was immersed in distilled water and the NaCl₂ extracted for 48 hr. The scaffolds were removed from the container and cut into desired dimensions.

For the preparation of Silk scaffolds embedded with antibiotic, 1 mg of antibiotic (gentamicin, cefazolin, and gentamicin/cefazolin in combination) was added to 2 ml of 6% (w/v) silk fibroin solution and the silk scaffold preparation procedures, as described herein, were followed.

To prepare Silk scaffolds embedded with antibiotic-loaded silk microspheres, 100 mg of 1,2-Dioleoyl-sn-glycero-3-phosphocholine (DOPC; Avanti Polar Lipids, Alabaster, Ala.) were dissolved in 1 ml chloroform in a glass tube and dried into a film under a flow of nitrogen gas. Wang et al., 117 J. Contr. Release 360-70 (2007). Two milligrams (2 mg) of the antibiotic (gentamicin, cefazolin, and gentamicin/cefazolin in combination; Sigma-Aldrich, St. Louis, Mo.) were mixed with 2 ml of 8% (w/v) silk fibroin solution and this mixture was added to hydrate the lipid film in installations of 0.33 ml, 0.5 ml, and 1 ml. The mixture was diluted to 2 ml with distilled water and moved to a plastic tube. The sample was frozen in liquid nitrogen for 15 min and then thawed at 37° C. for 15 min. The freeze-thaw step may help create smaller vesicles with homogeneous size distributions. Brandl, 7 Biotech. Ann. Rev. 59-85 (2001); Colletier et al., 2 BMC Biotech. 9 (2002). The freeze-thaw cycle was repeated three times and then the thawed solution was slowly pipetted into 50 ml water with fast stirring. The resulting solution was lyophilized for 72 hr and stored at 4° C.

Twenty milligrams (20 mg) of the lyophilized material was suspended in 2 ml of pure methanol in an Eppendorf tube, and the suspension was incubated for 30 min at room temperature. After the methanol induced (β-sheet structure self-assembly, the lipid templates (white viscous material) were removed and the mixture was centrifuged at 10,000 rpm for 5 min at 4° C. (Eppendorf 5417R centrifuge). The pellet obtained was dried in air and stored at 4° C. To generate a suspension of silk microspheres, the dried pellet was washed once with 2 ml of distilled water by centrifugation, and then re-suspended in water. The clustered microspheres were dispersed by ultrasonication for 10 sec at 30% amplitude (approximately 20 W) using a Branson 450 ultrasonicator (Branson Ultrasonics Co., Danbury, Conn.). The antibiotic-loaded microsphere suspension was added directly to 2 ml of 8% (w/v) silk fibroin solution and the silk scaffold preparation procedures, as described herein, were followed. See also WO 2008/118133. Micro- and nano-particles may also be prepared using phase separation of silk and polyvinyl alcohol without exposure to an organic solvent. See U.S. Patent Application Ser. No. 61/246,676.

For Silk scaffolds coated with antibiotic layers, the buildup of multiple layers of antibiotics on silk scaffolds was accomplished by the consecutive adsorption of silk fibroin and the antibiotic using a modified protocol previous reported. Wang et al., 21 Langmuir 11335-41 (2005). The silk scaffold preparation procedure, as described above, was followed and the dried scaffolds were dipped in 1 mg/ml of antibiotic solution (gentamicin, cefazolin, and gentamicin/cefazolin combination) for 3 min. The scaffolds were dried at 37° C. for 10 min and then dipped in a dilute 0.2% (w/v) silk fibroin solution for 3 min. The scaffolds were dried at 37° C. for 10 min and the coating process was repeated two times for a total of three antibiotic layers. See also WO 2007/016524.

To prepare Antibiotic-loaded electrospun silk fibroin scaffolds, electrospun silk fibroin scaffold mats were prepared as described previously and coated with antibiotic layers (gentamicin, cefazolin, and gentamicin/cefazolin combination) as previously described. Zhang et al., 29 Biomaterials 2217-27 (2008).

The antibiotic release and antimicrobial susceptibility experiments for each sample prepared here were examined as in Examples 3 and 4.

### Example 3. Antibiotic Release Experiments

Scaffolds containing antibiotics and controlled scaffolds containing no antibiotic were cut into cylinders of 6 mm diameter. The scaffolds were immersed in 3 ml distilled water and incubated at room temperature without shaking. At 24 hr intervals for 168 hr, 100 μl of each solution was withdrawn and the water replenished. The amount of antibiotic released was assayed spectrophotometrically (SPECTRAMAX® spectrophotometer, Molecular Devices, Sunnyvale, Calif.).

Cefazolin absorbs UV light at 270 nm. Voisine et al., 356 Int. J. Pharm. 206-11 (2008). Gentamicin does not absorb UV light, however, and thus o-phthaldialdehyde reagent (OPA; Sigma-Aldrich, St. Louis, Mo.) was used to analyze gentamicin concentration. Cabanes et al., 14 J. Liq. Chrom. 1989-2010 (1991); Chang et al., 110 J. Contr. Release 414-21 (2006).

One hundred microliters (100 μl) of the aqueous solution containing gentamicin was added to 100 μl isopropanol and 100 μl o-phthaldialdehyde reagent. The samples were incubated at room temperature for 45 min before measuring UV absorbance at 333 nm. For scaffolds prepared with the gentamicin/cefazolin combination, cefazolin detection was performed first followed by reaction with OPA for gentamicin quantification. The concentration of gentamicin and cefazolin was obtained by comparison with a calibration curve using a series dilution of the antibiotics in water. Background absorption of silk fibroin determined by control scaffolds was subtracted from experimental samples.

All assays were performed in triplicate and results reported as means±standard deviation. Significance levels were determined by ANOVA or by Student's t-test in SPSS Version 13.0 (SPSS, Inc., Chicago, Ill.). Differences were considered significant when p<0.05.

### Example 4. Antimicrobial Susceptibility Experiments

The susceptibilities of E. coli ATCC 25922 and S. aureus ATCC 25923 (both from American Type Culture Collection, Manassas, Va.) to the antibiotic-loaded scaffolds were determined by a modified Kirby-Bauer disc diffusion on agar method according to the National Committee for Clinical Laboratory Standards: Methods for dilution antimicrobial susceptibility testing for bacteria that grow aerobically Approved standard M7-A5, (Nat. Committee Clin. Lab. Standards, Wayne, Pa., 2000). One milliliter (1 ml) of each bacterial vial was suspended in 5 ml #18 Tryptic Soy Broth (Becton Dickenson, Sparks, Md.) and the density of the suspension was estimated to match the turbidity standard of 3.0×10⁸ CFU/ml (McFarland Standard, BioMerieux, Marcy l'Etoile, France). Mueller Hinton agar 100 mm plates were inoculated with 1 ml of bacterial suspension. The suspension was spread over the surface of the agar plates using a sterile 1 ml syringe and rod to ensure complete coverage.

Scaffolds containing antibiotics and control scaffolds containing no antibiotic were cut into cylinders of 6 mm diameter. Sterile forceps were used to place the scaffolds and SENSI-DISC™ antimicrobial susceptibility test discs (gentamicin, 10 μg; cefazolin, 30 μg; Becton Dickenson, Sparks, Md.) on the plates, spaced 15 mm from the edge of the plate and gently pressed to ensure even contact. The plates were incubated for 24 hr at 37° C. and zones of clearance (mm) were recorded.

All assays were performed in triplicate and results reported as means±standard deviation. Significance levels were determined by ANOVA or by Student's t-test in SPSS Version 13.0 (SPSS, Inc., Chicago, Ill.). Differences were considered significant when p<0.05.

### Example 5. Controlled Release of Penicillin and Ampicillin from Silk Films

Although longer term release profiles were studied for many of the material format studies, only the first 24 hours of penicillin and ampicillin release from silk films were characterized due to the literature reporting a 6-ht post-implantation “decisive period” during which implants are particularly susceptible to surface bacterial colonialization (Zilberman & Elsner, 130 Contr. Release 202-15 (2008)). The first 24 hrs of release from silk films were therefore considered most critical in prevention of bacterial adhesion and long-term implant success.

Release was determined using the previously described zone of inhibition assay in S. aureus lawns. Briefly, diluted Luria-Bertani (LB) agar was mixed with overnight S. aureus culture and added to LB agar plates. Standards and materials for testing were placed on the inoculated dilute agar layers and plates were grown overnight at 37° C. Zone of inhibition was measured the next day using Image J imaging software. Amount of drug released over 24 hr was then determined by comparison of sample zones of inhibition to the zones of inhibition of known standards on filter paper disks. Extended release studies were carried out by transferring the material being tested to new plates every 24 hr. Antibiotic-loaded silk films were prepared by mixing drug into 8% (w/v) silk solution and drying films overnight at ambient conditions. Two loading concentrations were studied: high loading (5 mg/mL, approx. 0.4 mg per film) and low loading (2.5 mg/mL, ˜0.2 mg per film). To assess the effects of methanol on incorporated antibiotics, films were either treated with methanol for 5 min or left untreated. Results of ampicillin and penicillin release from silk films are reported in Tables 1 and 2, respectively:

TABLE 1
Ampicillin Film Release

| | High | | Low | |
|---|---|---|---|---|
| | Loading/ | High | Loading/ | Low |
| | Methanol | Loading/ | Methanol | Loading/ |
| | treated | Untreated | treated | Untreated |
| Average active | 213.8 ± | 176.1 ± | 68.5 ± | 68.3 ± |
| ampicillin | 50.5 | 45.5 | 9.3 | 24.0 |
| Release (in μg) | | | | |
| Fraction of total | 0.53 | 0.44 | 0.34 | 0.34 |
| theoretical film | | | | |
| load released in | | | | |
| the first 24 hours | | | | |

High loading = 5 mg/mL ampicillin in an 8% (w/v) silk solution (theoretical load = 0.4 mg per film)
Low loading = 2.5 mg/mL ampicillin in an 8% (w/v) silk solution (theoretical load = 0.2 mg per film)

TABLE 2
Penicillin Film Release

| | High | | Low | |
|---|---|---|---|---|
| | Loading/ | High | Loading/ | Low |
| | Methanol | Loading/ | Methanol | Loading/ |
| | treated | Untreated | treated | Untreated |
| Average active | 345.9 ± | 167.1 ± | 194.0 ± | 205.9 ± |
| penicillin | 140.9 | 24.8 | 5.0 | 22.6 |
| Release (in μg) | | | | |
| Fraction of total | 0.43 | 0.21 | 0.48 | 0.51 |
| theoretical film | | | | |
| load released in | | | | |
| the first 24 hours | | | | |

High loading = 10 mg/mL penicillin in an 8% (w/v) silk solution (theoretical load = 0.8 mg per film)
Low loading = 5 mg/mL penicillin in an 8% (w/v) silk solution (theoretical load = 0.4 mg per film)

Results are reported as averages with standard deviations for n=3 films. Theoretical loading is calculated based on the concentration of drug in the silk solution and volume of solution used in casting the film. When methanol residue from methanol treatment was evaporated overnight, resuspended in PBS and assayed, no antibiotic activity was detected. These results suggest that methanol treatment does not degrade the incorporated antibiotic, as no reduction in activity is seen in the methanol treated films compared to the untreated films. The results also show that films deliver approximately half of their initial load within the first 24 hr of bacterial exposure.

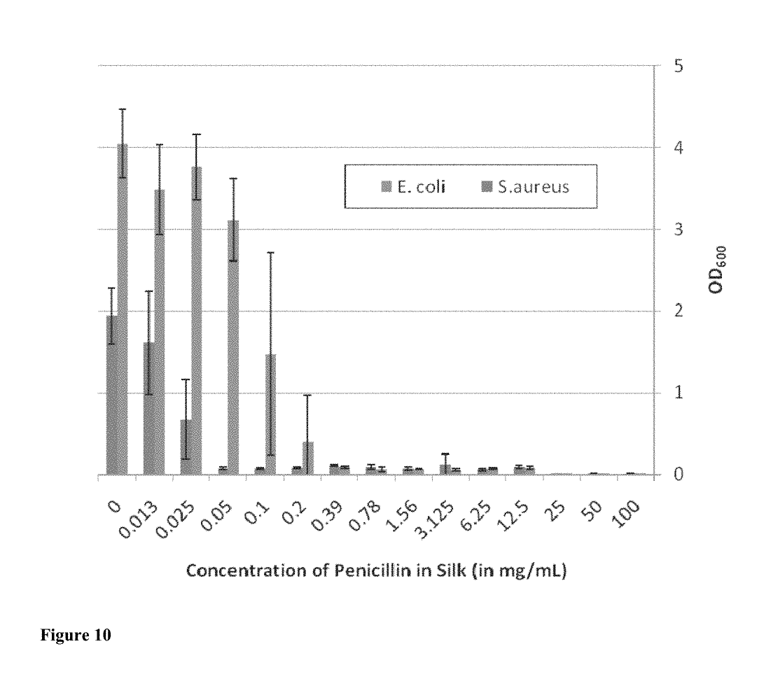

To determine the minimum inhibitory penicillin loading concentration in silk films, penicillin was mixed into an 8% silk solution in concentrations ranging from 100 mg/ml down to 0.013 mg/ml. 200 μl of silk+penicillin solution were aliquoted into each well of a 48-well plate, dried overnight at ambient conditions, methanol treated for 5 minutes and dried again. 400 μL of overnight S. aureus or E. coli culture diluted 1/100 in LB broth were added to each well. The cultures were grown at 37° C. for 48 hr, and then the optical density at 600 nm was measured for each sample with a UV spectrophotometer to determine bacterial growth. The results are shown in FIG. 10.

For both bacteria tested, total inhibition was seen in films prepared with 25 mg, 50 mg, or 100 mg or penicillin per ml of silk (5 mg, 10 mg and 20 mg of penicillin per film, respectively). Minimum concentrations were 0.39 mg/ml and 0.05 mg/ml required to induce near total inhibition in E. coli and S. aureus, respectively. These results suggest that, prepared in sufficient drug concentrations, these films can be used to prevent infection and totally suppress bacterial growth.

### Example 6. Antibiotic Delivery Via Bulk-Loaded Silk Hydrogels and Silk Microspheres Imbedded in Silk Hydrogels

Systems for injectable delivery of antibiotic releasing silk biomaterials were also studied. Drug release from both bulk loaded gels and gels loaded with drug releasing silk microspheres were characterized. Bulk loaded gels were prepared by sonicating silk solution using a Branson Digital Sonifier 450 at 15% amplitude for 60 sec to 90 sec, then mixing in antibiotic solution, then waiting for gelation to occur, thus entrapping the drug. Microspheres were prepared according to the lipid-template protocol described herein. As with the bulk loading, silk was sonicated and mixed with a microsphere suspension just prior to gelation.

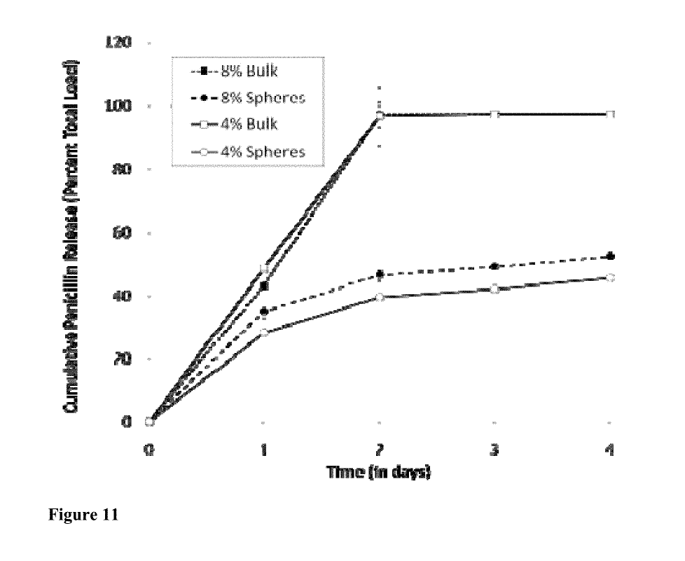

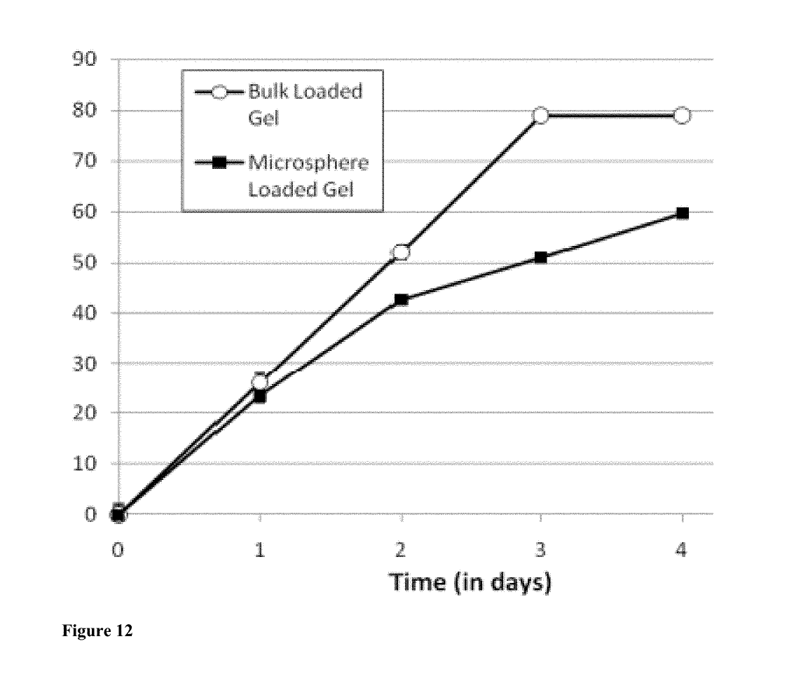

For penicillin-loaded gels, 8% (w/v) and 4% (w/v) silk hydrogel was studied to examine the effects of silk concentration on drug release. As no noticeable effect was observed, ampicillin release studies were carried out only using 8% (w/v) silk. Results of the zone of inhibition study in S. aureus lawns for penicillin loaded gels are shown in FIG. 11 and results of the ampicillin loaded silk gels are shown in FIG. 12.

Bulk-loaded gels exhausted their penicillin drug load within 48 hr and their ampicillin load within 72 hr. Microsphere-loaded gels released at a lower daily release rate than bulk loaded but continued to release for 4 days for both penicillin and ampicillin. Loading of drug into silk microspheres sustained release and delayed burst compared to bulk loaded gels. This suggests that bulk loading could be used to deliver a large initial burst dose, with microspheres incorporated for sustained release of a lower maintenance antibiotic dose. These results also show that injectable formats can effectively deliver active antibiotics.

### Example 7. Stabilization of Temperature-Sensitive Antibiotics in Silk Films

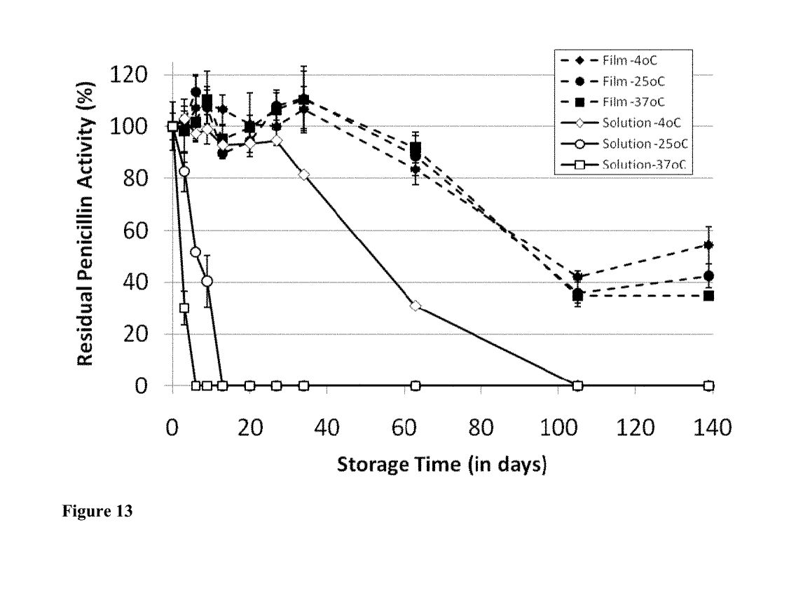

To determine if incorporation into silk films would have a similar stabilizing effect to that observed for enzymes, a long-term stability study was carried out comparing the residual activity of penicillin stored in 8% (w/v) silk films or in solution at 4° C. (refrigeration), 25° C. (room temperature) and 37° C. (body temperature). The results of the first five months of stability data are shown in FIG. 13.

Although stability rapidly declines for penicillin in solution stored at 25° C. and 37° C., temperature appears to have little effect on stability in silk films. Activity was still present after 140 days of storage. Note that for the first 40 days of storage, incubation enhanced activity above 100% of the initial value, a phenomenon also observed for enzyme storage data.

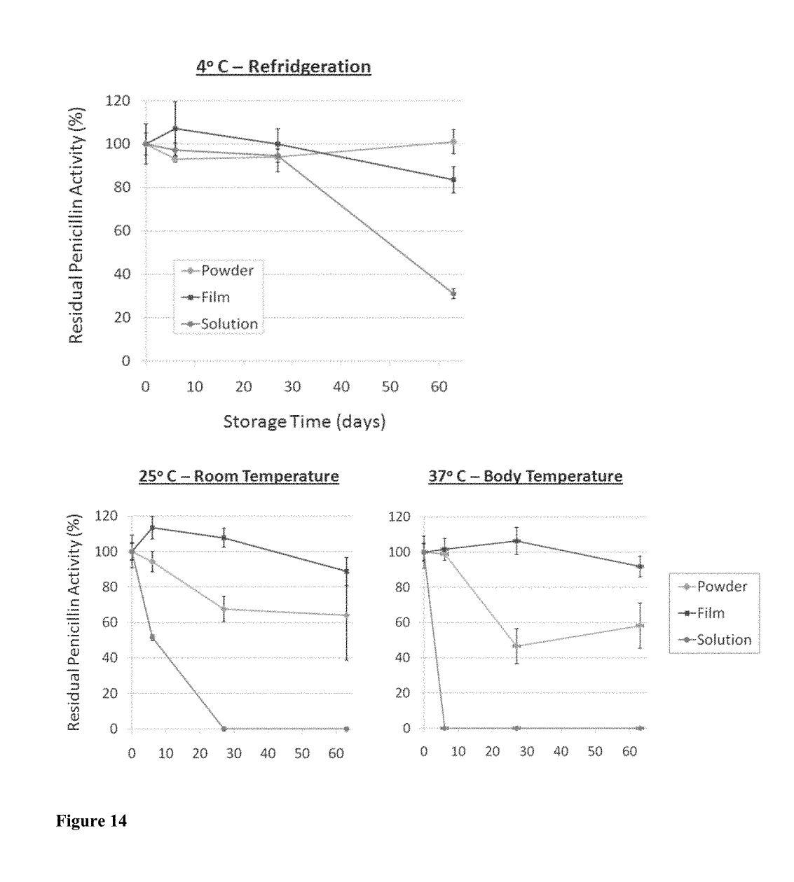

Stability was also characterized for dry penicillin powder stored at 4° C., 25° C., and 37° C. Comparison of the stability of penicillin stored in silk films, in solution and in dry powder format over 60 days is shown in FIG. 14.

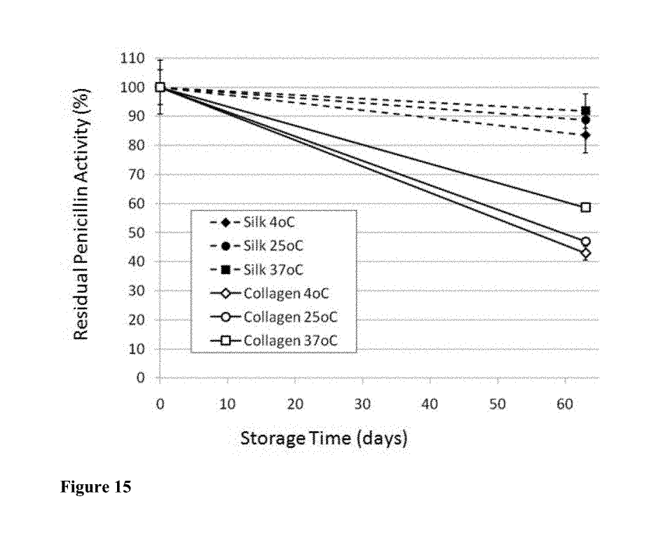

Dry powder and silk film performed equally well as penicillin stabilizers when stored at 4° C., but for samples stored at 25° C. and 37° C., activity was greater for penicillin in silk films compared with dry penicillin powder. Comparison of activity in collagen films of comparable drug loading and mass stored at various storage temperatures for 60 days (FIG. 15) also shows that activity is greater for penicillin stored in silk films compared with penicillin stored in collagen films.

### Example 8. Nanofilm Coatings of Gentamicin and Cefazolin on Porous Silk Scaffolds

Layer-by-layer nanofilm coating was studied as a potential loading strategy for water-soluble antibiotics and porous three-dimensional substrates. Prior to coating, the average mass of silk scaffolds was 25.1 mg±3.4 mg for the gentamicin coated scaffolds and 25.0 mg±4.8 mg for cefazolin coated scaffolds.

The nanofilm coating procedure described herein was followed, except that 8 mm diameter sponges were used rather than 6 mm, for the final layer of silk a higher concentration 4% (w/v) silk solution was used to provide a thicker capping layer and the drying times were closer to 30 min than 10 min.

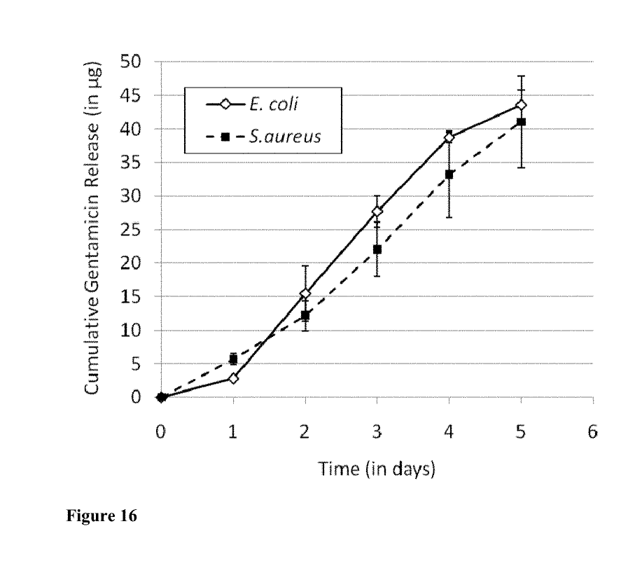

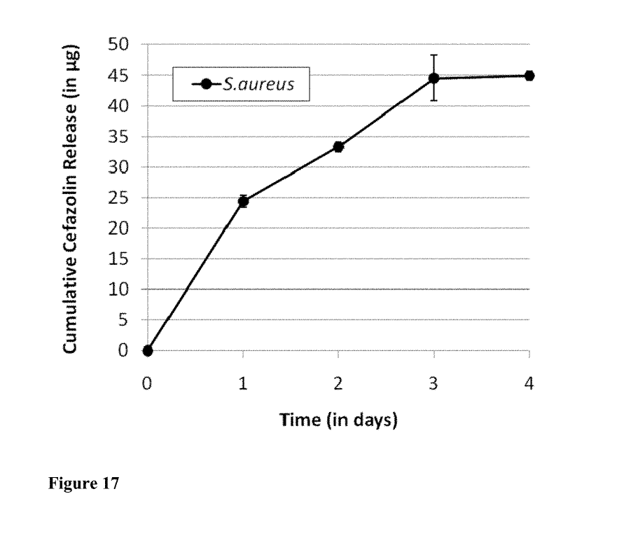

Cefazolin release data determined on S. aureus plates is shown. Gentamicin release data is shown based on both S. aureus and E. coli inhibition. In the end, gentamicin release duration was 5 days, cefazolin was 4 days. Data for cumulative gentamicin release is reported in Table 3 and shown in FIG. 16; cumulative cefazolin release is reported in Table 4 and shown in FIG. 17.

TABLE 3
Cumulative Release of gentamicin as determined by zone of
growth inhibition on E. coli and S. aureus lawns

| | Cumulative Gent Release | Cumulative Gent Release |
|---|---|---|
| Time (days) | (in μg) E. coli | (in μg) S. aureus |
| 0 | 0 | 0 |
| 1 | 2.81 | 5.70 |
| 2 | 15.46 | 12.20 |
| 3 | 27.71 | 22.05 |
| 4 | 38.70 | 33.21 |
| 5 | 43.57 | 41.08 |

TABLE 4
Cumulative release of cefazolin as determined by
zone of growth inhibition on S. aureus lawns

| | Cumulative Cefazolin Release |
|---|---|
| Time (days) | (in μg) S. aureus |
| 0 | 0 |
| 1 | 24.42 |
| 2 | 33.30 |
| 3 | 44.51 |
| 4 | 44.94 |

Total loading was determined by degrading finished coated scaffolds in 0.1 mg/mL proteinase k solution at 37° C. overnight. For gentamicin-loaded scaffolds, total loading was found to be 107.5 μg±30.95 μg (n=4 samples) and for cefazolin-loaded scaffolds, total loading was found to be 55.5 μg±6.98 μg (n=3 samples). The gentamicin-loaded scaffolds released between 40 μg and 45 μg over 5 days and the cefazolin-loaded scaffolds released ˜45 μg over 3 days. These results demonstrate that nanofilm coating can be used to achieve fairly linear, sustained release of water-soluble drugs from porous substrates to produce antibiotic-releasing silk sponges.

We claim:

1. A composition comprising antibiotic-loaded silk fibroin microspheres embedded in a three-dimensional silk fibroin scaffold.
